(19) United States
(12) Patent Application Publication
(10) Pub. No.: US 2018/0161375 A1
(43) Pub. Date: Jun. 14, 2018

(54) Therapeutic method for repairing damaged pancreas

(71) Applicant: Numen Biotech Co., Ltd, Taipei City (TW)

(72) Inventors: Shaw-Yih Liou, Taipei City (TW); Chih-Yang Huang, Taichung City (TW); Tsai-Jui Liou, Taipei City (TW); Tung-Sheng CHEN, Taichung City (TW); I-Te Liou, Taipei City (TW)

(21) Appl. No.: 15/889,703

(22) Filed: Feb. 6, 2018

Related U.S. Application Data
(63) Continuation-in-part of application No. 15/160,519, filed on May 20, 2016.

(30) Foreign Application Priority Data
Nov. 3, 2017 (TW) 106138102

(51) Int. Cl. A61K 35/28; A61K 31/353; A61P 5/00

(52) U.S. Cl. CPC A61K 35/28; A61P 5/00; A61K 31/353

(57) ABSTRACT

The invention is a therapeutic method for repairing damaged pancreas. The method comprises administrating Epigallocatechin gallate (EGCG) of green tea and a plurality of adipose-derived stem cells (ADSC). And Epigallocatechin gallate (EGCG) of green tea enhances the ability of the adipose-derived stem cells to repair damaged tissue.

### CROSS-REFERENCE TO RELATED APPLICATIONS

This application is a Continuation-in-Part of co-pending application Ser. No. 15/160,519, filed on May 20, 2016, for which priority is claimed under 35 U.S.C. § 120; and this application claims priority of Application No. 106138102 filed in Taiwan on Nov. 3, 2017 under 35 U.S.C. § 119; the entire contents of all of which are hereby incorporated by reference.

### FIELD OF THE INVENTION

The present invention is related to a therapeutic method for repairing pancreas injuries, particularly with providing a plurality of pretreated adipose-derived stem cells (ADSC) and epigallocatechin gallate (EGCG) of green tea. And EGCG of green tea can enhance the ability of the adipose-derived stem cells for repairing damaged tissue.

### BACKGROUND OF THE INVENTION

The clinical determination of diabetes is that the fasting blood-glucose value is higher than 110 mg/dl and the glycated hemoglobin HbA1c value is higher than 6.5%. The cause of hyperglycemia is something wrong in the metabolic mechanism of blood glucose. The insulin secreted from pancreas is the foremost protein for metabolizing blood glucose, so the hyperglycemia is caused by abnormal insulin secretion.

In terms of Type I diabetes, the secretory volume of insulin is insufficient, a small amount of insulin cannot metabolize a large amount of blood glucose, high-concentration glucose accumulates in blood, forming hyperglycemia. Since the primary cause of Type I diabetes is hypoinsulinism, the Type I diabetes is mostly caused by dysfunction of pancreas. There are many factors in forming Type I diabetes, such as pancreatic cell death caused by autoimmune disease, pancreas hypofunction resulted from heredity problems or pancreas function damaged by virus infection, these factors can fail the pancreas function and cause insufficient secretory volume of insulin, the β cell of pancreas is the insulin producer in hysinsulinism. Previous findings on mice showed that if the β cells of injured pancreas were repaired, the volume of insulin increased relatively, and the blood-sugar content returned to normal value.

In terms of the management of Type I diabetes at present, besides insulin injection and pancreas transplantation, there are control of habits and customs (e.g. regular exercise, low-calorie low-oil diet, body weight control) and pharmacotherapy. Besides the aforesaid methods, some documents mentioned transplanting stem cells to treat diabetes. However, treating diabetes by transplanting stem cells is mostly seen in fundamental medical research reports, and there are few documents about clinical treatment of diabetes by transplanting stem cells.

In addition, it is indicated that a high glucose environment injures stem cells, degrading the mobility or restorability of cells, even increasing the oxidizing pressure in stem cells. In other words, how to let the stem cells exist in an inappropriate environment, such as a high glucose environment, and repair the damaged organs effectively, such as pancreas, is an important topic.

Hence, how to let the stem cells exist in unfavorable environments for cell growth, such as high glucose and high oxidizing pressure microenvironments; to further improve and treat the damaged organs of organisms, and to solve the deficiencies in the known techniques are urgent issues for related technical fields to solve.

### SUMMARY OF THE INVENTION

Accordingly, the present invention provides the method for repairing a damaged pancreas in a subject, wherein the method comprising administrating to said subject a plurality of pretreated adipose-derived stem cells and an ester type catechin, wherein the plurality of pretreated adipose-derived stem cells are cultured with the ester type catechin.

Preferably, the damaged pancreas causes diabetes or hyperglycemia.

Preferably, the damaged pancreas is a plurality of damaged beta cells.

Preferably, the pretreated adipose-derived stem cells are pretreated with the ester type catechin for 2 hours.

Preferably, the concentration of ester type catechin for the pretreated adipose-derived stem cells is 5-25 μM.

Preferably, the concentration of ester type catechin for the pretreated adipose-derived stem cells is lower than 20 μM.

Preferably, the pretreated adipose-derived stem cells have higher viability in a high glucose environment, wherein the high glucose environment means the glucose concentration is 22 mM-44 mM, mainly 33 mM, the high viability is 80%˜100%.

Preferably, the pretreated adipose-derived stem cells increase expression level of a group of survival related protein in the damaged pancreas, wherein the group of survival related protein comprising p-IGF1R, PI3K, p-PI3K, Akt, p-Akt, p-Bad and Bcl-xL, the percentage of increase is 50%˜100%.

Preferably, the pretreated adipose-derived stem cells increase expression level of a group of Sirt1 pathway related protein in the damaged pancreas, wherein the group of Sirt1 pathway related protein comprising p-AMPK and Sirt1, the percentage of increase is 50%˜100%.

Preferably, the pretreated adipose-derived stem cells reduce expression level of a group of cell apoptosis pathway related protein in the damaged pancreas, wherein the group of cell apoptosis pathway related proteins comprising FADD, Caspase 8, Bax, Cytochrome C and Caspase 3, the percentage of decrease is 40%˜80%.

Preferably, the pretreated adipose-derived stem cells reduce expression level of a fibrosis related protein in the damaged pancreas, wherein the fibrosis related protein is TGF-β, the percentage of decrease is 40%˜80%.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### BRIEF DESCRIPTION OF THE DRAWINGS

FIG. 1A shows the analysis of stem cell characteristics according to the stem cell surface marker;

FIG. 1B shows the analysis of stem cell characteristics by stem cell differentiation potency;

FIG. 2 shows (a) cell viability analysis; (b) survival-related protein expression level analysis of stem cells in high glucose environment;

FIG. 3 shows the analysis of blood glucose values of experimental animal before and after stem cell treatment;

FIG. 4 shows the experimental animal pancreatic tissue survival related protein expression level analysis and Western Blot analysis;

FIG. 5 shows the experimental animal pancreatic tissue survival-related protein expression level analysis and quantification;

FIG. 6 shows the experimental animal pancreatic tissue Sirt1 pathway related protein expression analysis and Western Blot analysis;

FIG. 7 shows the experimental animal pancreatic tissue Sirt1 pathway related protein expression analysis and quantification;

FIG. 8 shows the experimental animal pancreatic tissue apoptosis pathway related protein expression analysis and Western Blot analysis;

FIG. 9 shows the experimental animal pancreatic tissue apoptosis pathway related protein expression analysis and Western Blot analysis;

FIG. 10 shows the experimental animal pancreatic tissue apoptosis pathway related protein expression analysis and quantification;

FIG. 11 shows the experimental animal pancreatic tissue apoptosis pathway related protein expression analysis and quantification;

FIG. 12 shows the experimental animal pancreatic tissue slice analysis and HE staining analyzed insular tissue size;

FIG. 13 shows the experimental animal pancreatic tissue slice analysis and insular tissue size analysis quantification;

FIG. 14 shows the experimental animal pancreatic tissue slice analysis and Masson's Trichrome staining analyzed pancreas tissue fibrosis;

FIG. 15 shows the experimental animal pancreatic tissue slice analysis and Western Blot analyzed pancreatic tissue fibrosis related protein expression.

### DETAILED DESCRIPTION OF THE INVENTION

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

The present invention provides a therapeutic method for treating pancreas injuries. The therapeutic method administrating to said subject a plurality of pretreated adipose-derived stem cells and an ester type catechin.

The following embodiments are provided for illustrative description, not to limit the scope of the present invention.

### Example 1. The Preparation of Animal Experiments Thereof

1. Animal Experiment Design

The 8 months old Wistar strain male rats (bought from Lasco company) are divided into four groups, which are normal group (Sham), STZ (55 mg/kg) induced diabetes group (DM), diabetes+autogenous ADSC treatment group (DM+ADSC) and diabetes+green tea EGCG pretreated autogenous ADSC treatment group (DM+E-ADSC), as shown in Table 1. The rats are bred in the animal house for 12 hours during the day and 12 hours at night circularly. The food and water are taken freely during breeding. Two rats live in one animal cage, the animal litter is changed every two days during breeding. When the blood glucose of the rats of diabetes groups increase to 200 mg/dl, the diabetes is determined. The determined diabetes groups are treated by transplanting autogenous stem cells one month later. In terms of feedback of autogenous stem cells, 1×10⁵˜1×10⁷ stem cells are fed back into each rat through vena caudalis.

TABLE 1
Animal experiment grouping

| Sham | DM | DM + ADSC | DM + E-ADSC |
|---|---|---|---|
| Normal | Diabetes | Diabetes + stem cell | Diabetes + EGCG pretreated |
| group | group | treatment group | stem cell treatment group |

2. Blood Glucose Analysis

For the rat blood glucose test, the fasting caudal vein blood is tested by Roche blood glucose meter (Accu-Chek Active).

### Example 2. The Preparation of Rat ADSC Extraction and Stem Cell Characteristic Analysis

1. ADSC Extraction

The adipose tissue in the abdominal cavity of an 8 months old Wistar strain male rat is extracted surgically. The adipose tissue is cut in appropriate size, cleaned with physiological saline solution containing antibiotics, the cleaned adipose tissue is put in the physiological saline solution containing Type II collagenase (0.01%), heated and stirred in 37° C. water bath for about 1 hour, centrifugated at 3000 rpm at room temperature for about 10 minutes, the lower precipitate is extracted and put in the cell culture dish for cell culture.

2. Stem Cell Characteristic Analysis

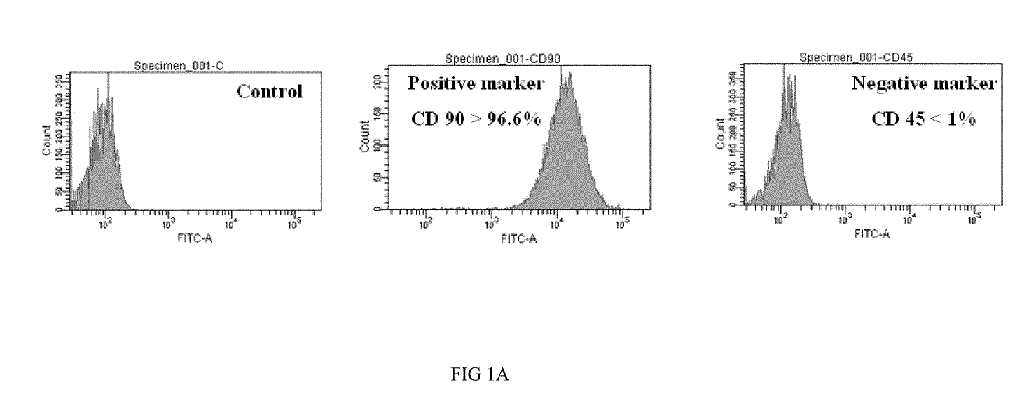

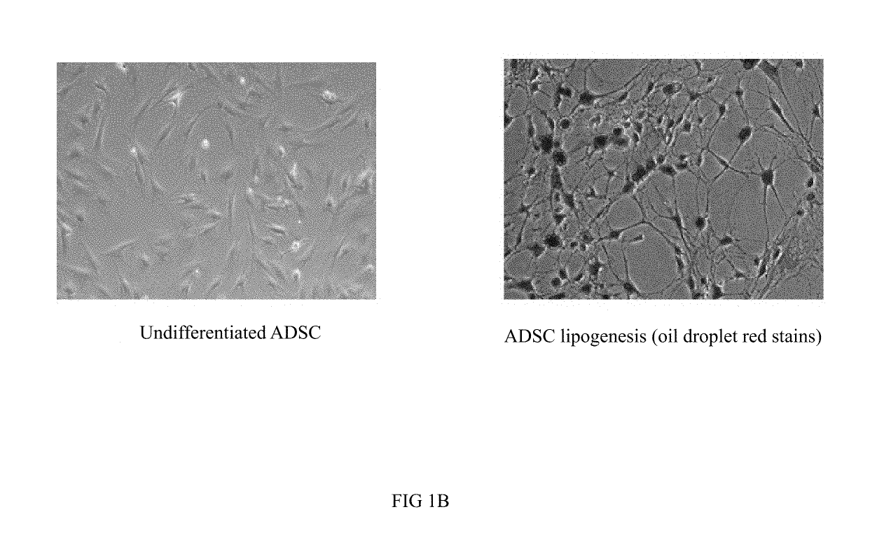

Before the autotransplantation of stem cells for treatment, it is required to confirm whether the transplanted cells are stem cells or not. There are two kinds of analysis mode to determine stem cells. The first mode uses flow cytometer to analyze the positive marker and negative marker on the stem cell membrane. The characteristic of stem cells is that the higher the expression level of positive marker is, the lower is the expression level of negative marker. The other mode is differentiation experiment, to observe whether the stem cells are able to be differentiated into other heterogeneous cells. According to FIG. 1 (A), the expression level of positive marker CD90 of cell mass is 96.6%, and the expression level of negative marker CD45 is 1%. In addition, FIG. 1 (B) shows this cell mass is able to be differentiated into adipocytes. To sum up, this cell mass has the characteristics of stem cells.

### Example 3. The Analysis of Cell Viability

The cells are cultured in 24-well dish, the cells are medicated, the culture medium is removed, washed with PBS buffer (three times), cultured in culture medium containing 0.5 mg/ml MTT for 3˜4 hours, the culture medium is removed, washed with PBS buffer, mixed with 1 ml isopropanol to dissolve purple formazan crystal, the O.D.570 nm absorbance value determination is implemented 5 minutes later.

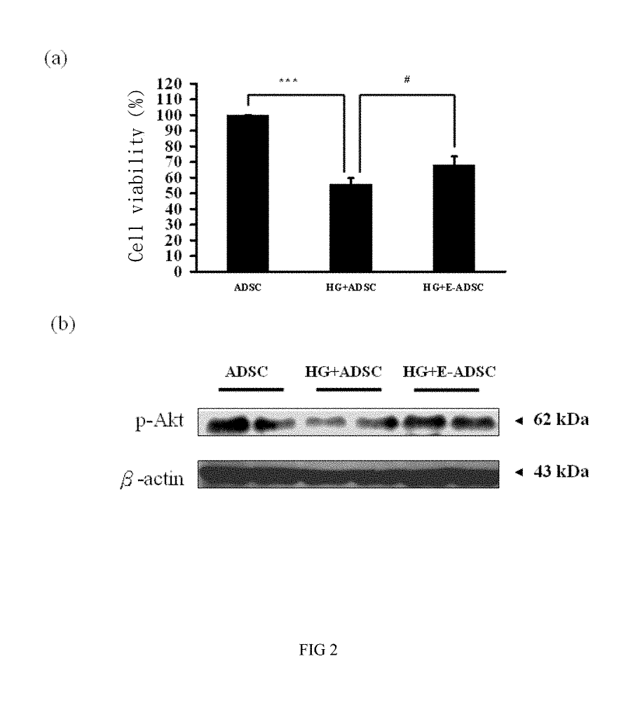

The cell viability of stem cells under the stimulation of high glucose environment is shown in FIG. 2 (a). Compared with control group (ADSC), the viability of the stem cells in high glucose environment (HG+ADSC) is 56±4% (p<0.001). If the stem cells are pretreated with EGCG and stimulated by high glucose (HG+E-ADSC), the viability is 68±5%, apparently higher than HG+ADSC group (HG+E-ADSC >HG+ADSC, p<0.05).

### Example 4. The Analysis of Protein Concentration Determination

The protein is quantified by Bradford protein assay. The principle is that the protein and Coomassie brilliant blue G-250 can form a blue compound. The darker the blue is, the higher is the protein content. In terms of the test method, a series of BSA at given concentration is mixed with 1/5 Bradford protein dye by volume, a standard curve is made from the brightness of 595 nm visible light, and the O.D. value of sample is measured in the same way, the concentration of sample protein can be obtained according to the standard curve.

### Example 5. The Analysis of Blood Glucose of Rat

For the rat blood glucose test, the fasting caudal vein blood is tested by Roche blood glucose meter (Accu-Chek Active).

Changes in blood glucose values of various groups of experimental animals before and after stem cell treatment

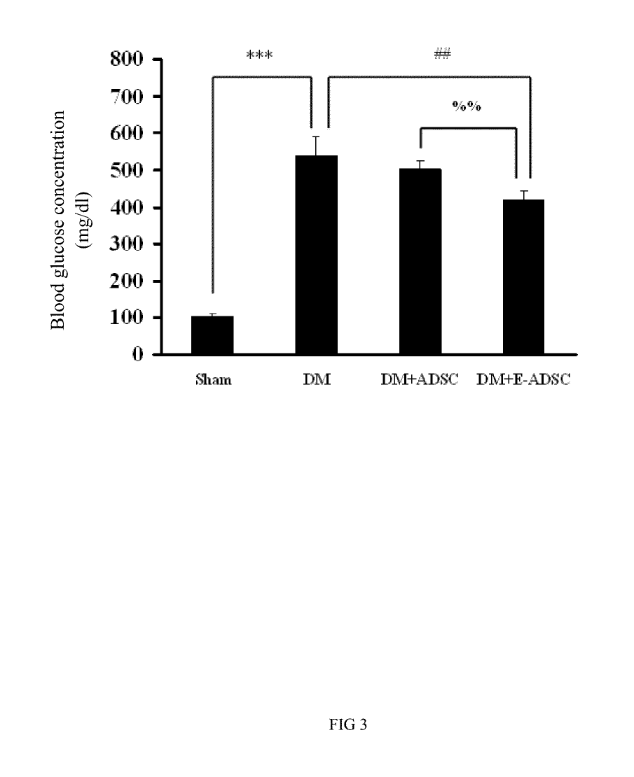

FIG. 3 shows the analysis of blood glucose values of experimental animals before and after stem cell treatment. It is observed that the blood glucose values of normal group (Sham), diabetes group (DM), diabetes+stem cell treatment group (DM+ADSC) and diabetes+EGCG pretreated stem cell treatment group (DM+E-ADSC) are 103±10, 540±50, 504±23 and 412±28 mg/dl respectively. The blood glucose value of DM group is apparently higher than that of Sham group (DM>Sham, p<0.001), and that of DM+E-ADSC group is apparently lower than that of DM group and DM+ADSC group (DM >DM+E-ADSC, p<0.01; DM+ADSC >DM+E-ADSC, p<0.01).

### Example 6. Western Blot

An amount of 40 ug protein sample is mixed with PBS solution and 5× loading dye uniformly and boiled for 10 minutes before SDS-polyacrylamide slab gel electrophoresis analysis. The upper colloid of SDS-polyacrylamide slab gel electrophoresis is 3.75% Stacking gel, the lower colloid is 5% and 12% Separating gel. The prepared slab gel is fixed to the electrophoretic apparatus, and the electrophoresis tank is filled up with electrophoretic buffer (Electrode buffer). Afterwards, the treated protein sample solution is poured into the U-shaped channel formed in the slab gel, electrophoresed with 75V. The protein transfer is implemented after electrophoresis, the colloid is extracted, the colloid is laid on a wetted Whatman 3M filter paper. The colloid is covered with a methanol wetted PVDF membrane, and covered with a wetted 3M filter paper, and it is put in Transfer Holder after the intermediate bubbles are swept slightly by a glass rod, and then it is put in Electrotransfer Tank (containing Transfer buffer) at 4° C., after 1-hour transfer under 100 V, the PVDF membrane is taken out and immersed in 5% (w/v) skimmed milk (Blocking buffer)(PBS-non-fat milk powder) and shaken at room temperature for one hour. The PVDF membrane is put in a 4° C. refrigerator, reacting with primary antibody overnight, and then cleaned with washing buffer twice, 10 minutes each time. It is dumped after the last cleaning. It reacts with horseradish peroxidase conjugated secondary antibody for 2 hours, the PVDF membrane is cleaned in the same way. Finally, the PVDF membrane is immersed in 4 ml substrate buffer for color reaction.

FIG. 2 (b) shows the survival-related protein p-Akt expression level when the stem cells are in high glucose environment, the high glucose environment scales down the stem cell survival-related protein p-Akt expression level obviously compared with ADSC (HG+ADSC<ADSC). The survival-related protein p-Akt expression level of the EGCG pretreated stem cells in high glucose environment (HG+E-ADSC) is apparently higher than HG+ADSC group.

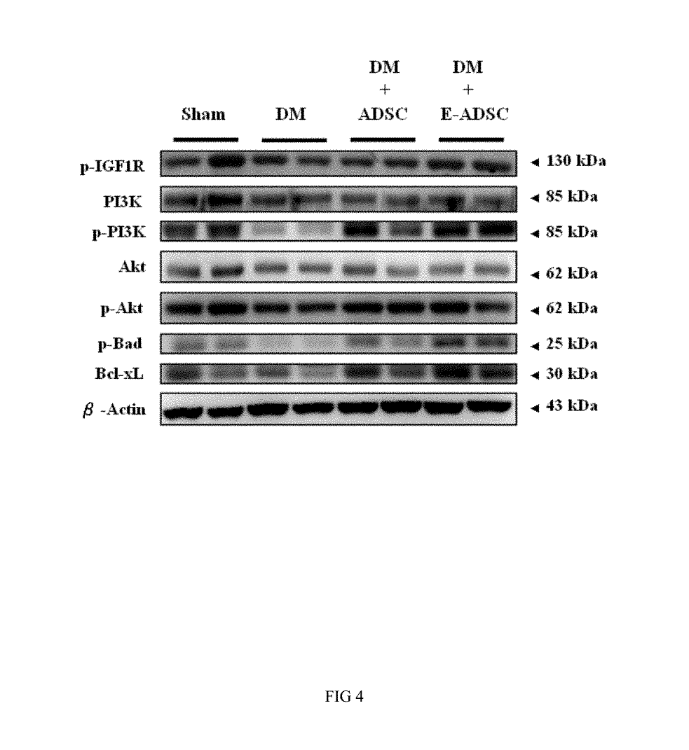

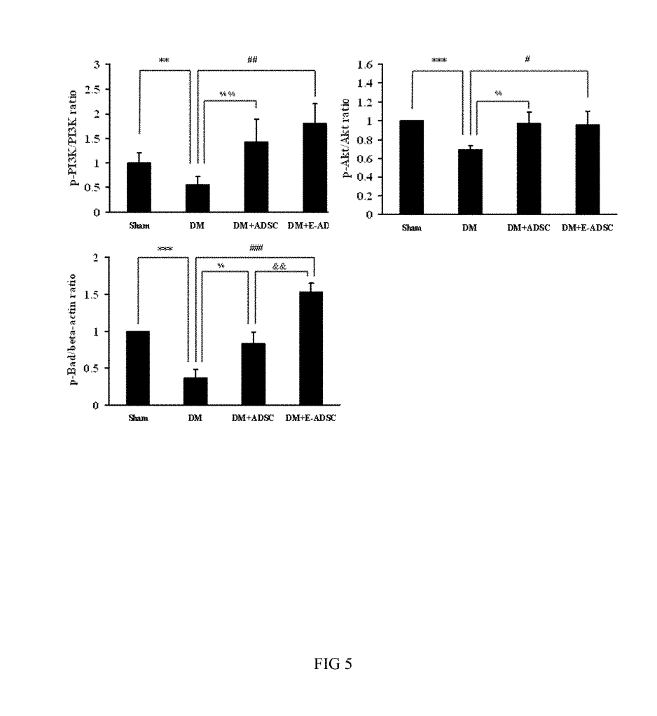

FIG. 4-5 shows the analysis of survival-related protein expression level of experimental animal pancreatic tissue. Compared with normal group (Sham), the diabetes group (DM) scales down the survival-related protein expression level of animal pancreatic tissue, including p-IGF1R, PI3K, p-PI3K, Akt, p-Akt, p-Bad and Bcl-xL. The survival-related protein expression level increases again after the treatment by stem cell transplantation (DM+ADSC and DM+E-ADSC). The EGCG pretreated stem cell treatment group (DM+E-ADSC) scales up the survival-related protein expression level most significantly.

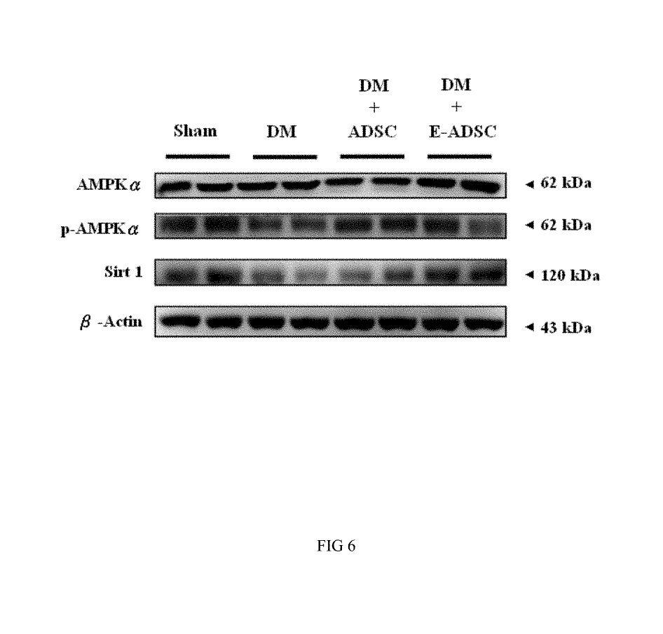

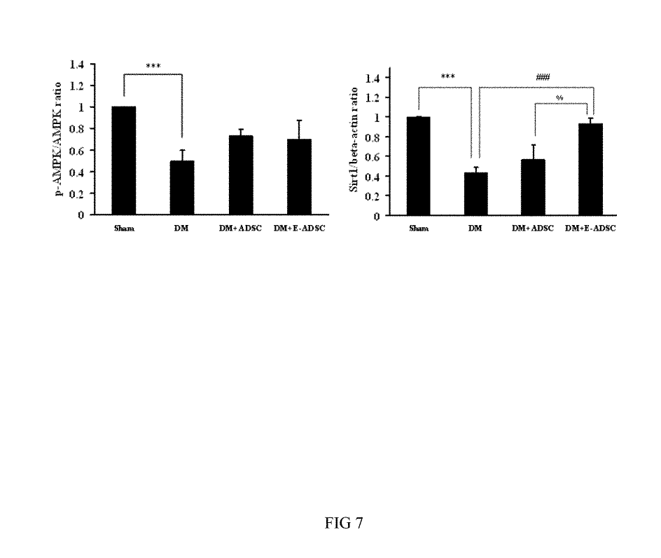

According to FIGS. 6-7, compared with normal group (Sham), the diabetes group (DM) scales down the Sirt1 pathway related protein expression of animal pancreatic tissue, including p-AMPK and Sirt1. The Sirt1 pathway related protein expression increases again after the treatment by stem cell transplantation (DM+ADSC and DM+E-ADSC), the EGCG pretreated stem cell treatment group (DM+E-ADSC) scales up the survival-related protein expression level most significantly.

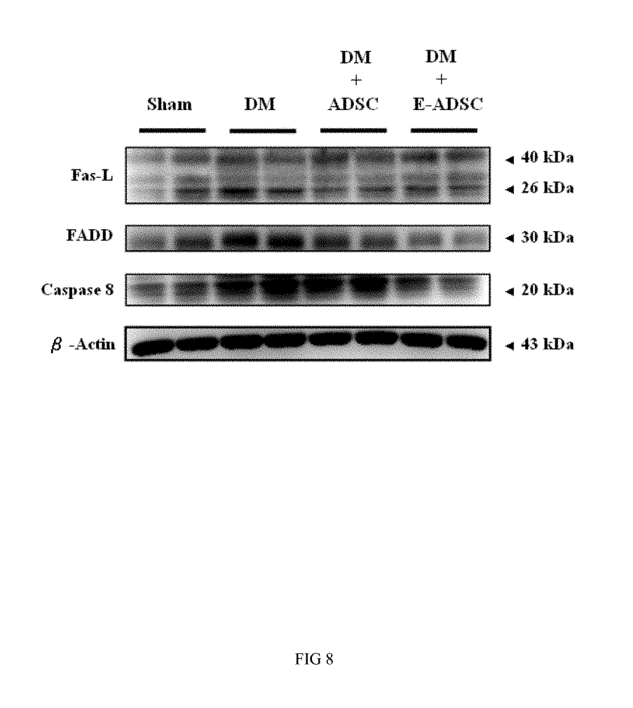

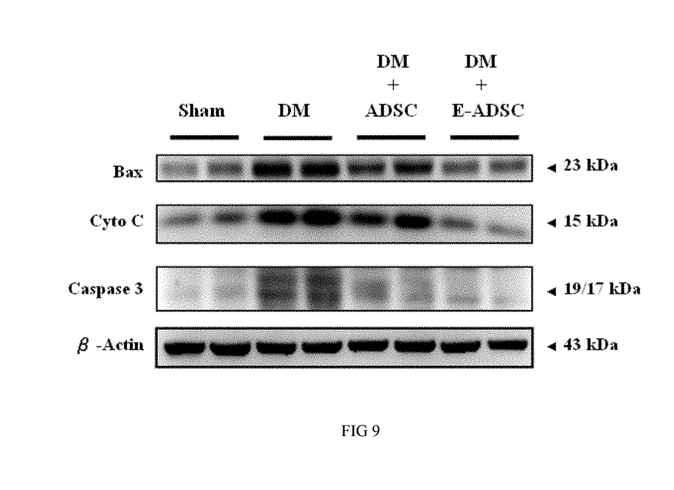

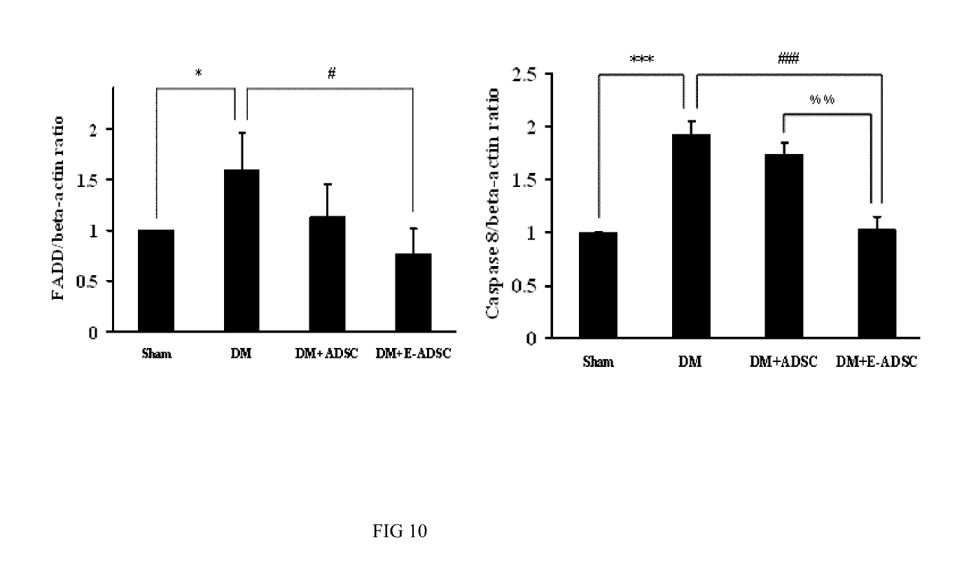

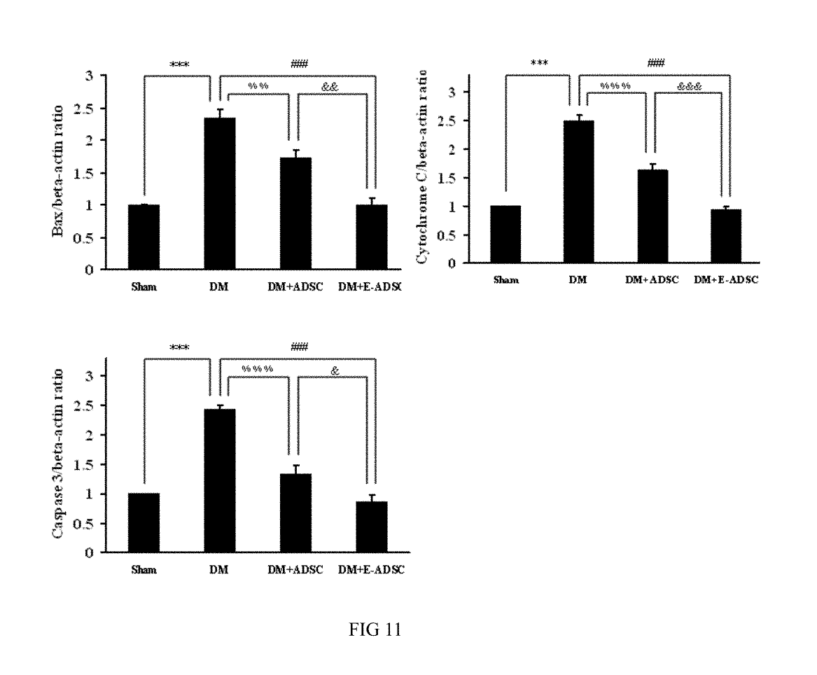

According to FIG. 8-11, compared with normal group (Sham), the diabetes group (DM) scales down the cell apoptosis pathway related protein expression of animal pancreatic tissue. The cell apoptosis pathway related protein expression decreases after the treatment by stem cell transplantation (DM+ADSC and DM+E-ADSC), the EGCG pretreated stem cell treatment group (DM+E-ADSC) scales up the survival-related protein expression level most significantly.

### Example 7. Section Staining

The pancreatic tissue is taken from the sacrificed rat, soaked in formalin to fix the tissue, and then dehydrated by ethanol, the pancreatic tissue is embedded in paraffin and cut up into 0.2 μm tissue slice. In HE staining, the tissue slice is dewaxed and fixed by ethanol and formalin, stained by hematoxylin and eosin for observation. In Masson's Trichrome staining, the tissue slice is dewaxed and fixed by ethanol and formalin, and stained by the following stains, Bouin's stain 15 minutes, Weiger stain 10 minutes, Beibrich stain 5 minutes, PPA stain 10 minutes and Aniline blue stain 5 minutes.

1. Experimental Animal Pancreatic Tissue Slice Analysis

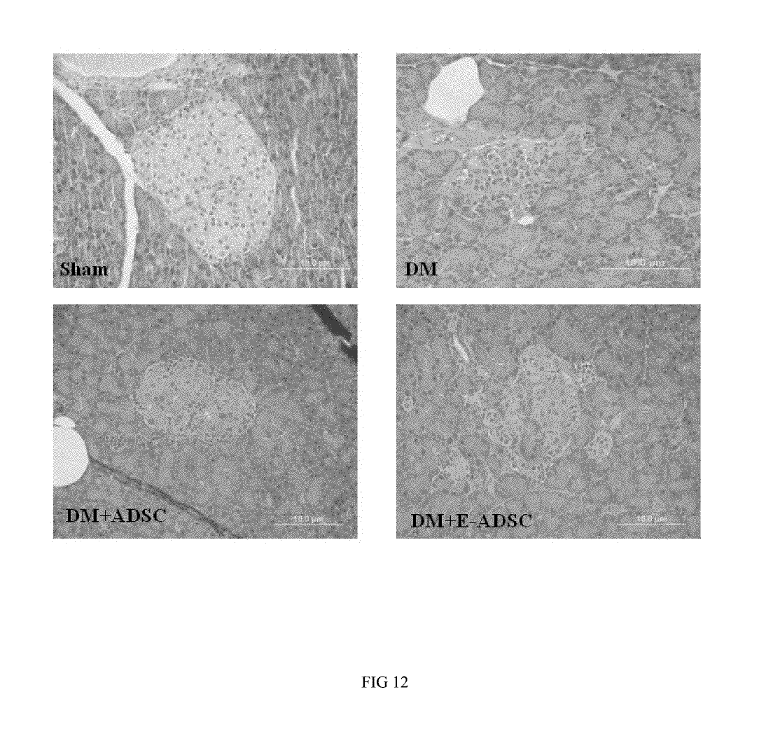

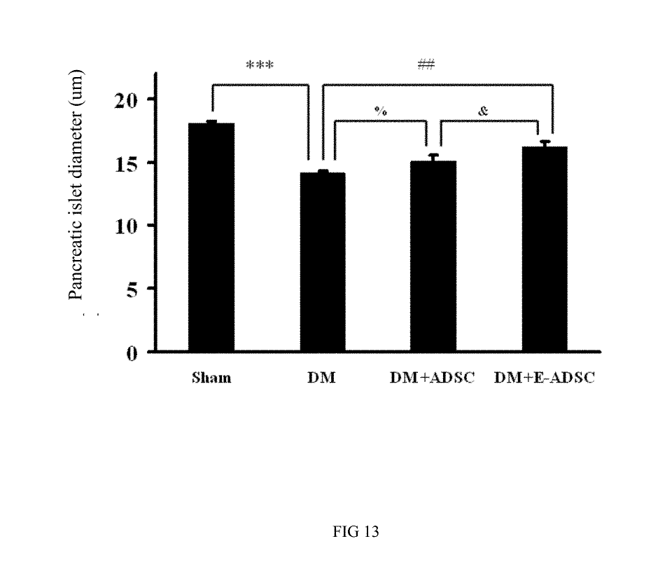

FIGS. 12 and 13 show the animal pancreatic tissue HE staining, the insular tissue diameter and the quantized diameter. It is observed that the insular tissue diameters of normal group (Sham), diabetes group (DM), diabetes+stem cell treatment group (DM+ADSC) and diabetes+EGCG pretreated stem cell treatment group (DM+E-ADSC) are 18±0.2, 14.1±0.2, 15.1±0.5 and 16.2±0.4 μm respectively. The insular tissue diameter of DM group is apparently smaller than that of Sham (DM<Sham, p<0.001), the insular tissue diameter increases obviously after the stem cell treatment (DM+ADSC>DM, p<0.05; DM+E-ACSC>DM, p<0.01), and the EGCG pretreated stem cells have better treatment effect than simple stem cell treatment (DM+ADSC <DM+E-ADSC, p<0.05).

2. Analysis of Experimental Animal Pancreatic Tissue Slice Fibrosis

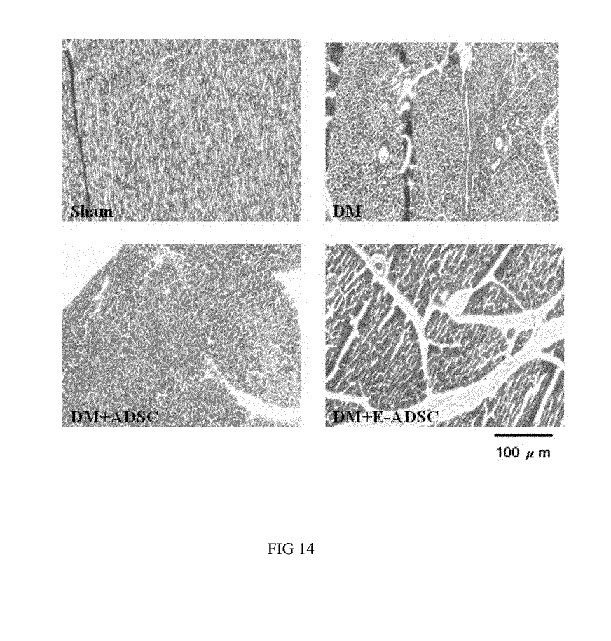

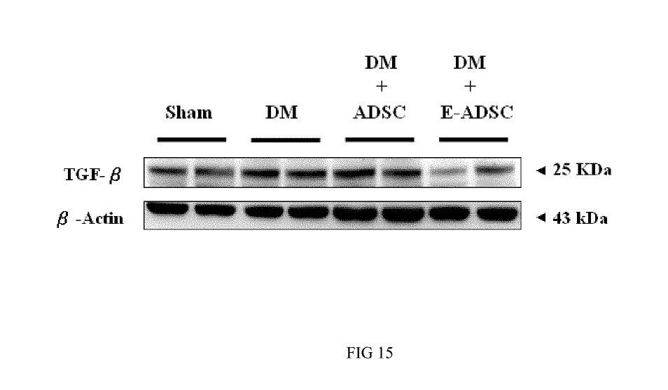

FIG. 14 shows the Masson's Trichrome staining of animal pancreatic tissue, the insular tissue fibrosis is observed. It is observed that the diabetes group (DM group) has more blue collagen stains than normal group (Sham group), representing severe fibrosis. The stem cell treatment groups DM+ADSC and DM+E-ADSC have less blue collagen stains, especially the DM+E-ADSC group. FIG. 15 shows the analysis of animal pancreatic tissue fibrosis related protein expression analysis. Compared with Sham group, the TGF β protein expression of DM group is markedly increased. The TGF β protein expression of stem cell treatment groups DM+ADSC and DM+E-ADSC decreases apparently, especially the DM+E-ADSC group.

3. Statistical Analysis

The analysis results of each specimen/sample are obtained by analyzing the data of three or more experiments. The ANOVA is used for statistics, p<0.05 represents significant difference.

According to the experimental results, the stem cell treatment can reduce the blood glucose of the rat with diabetes, the mechanism is to scale up the pancreatic tissue survival related protein, scale up the Sirt1 pathway related protein, scale down the expression level of apoptosis pathway related protein, the size of insular tissue of pancreas is restored, so as to restore the pancreas function, and the blood glucose value is scaled down. Meanwhile the stem cell treatment can scale down the fibrosis related protein expression in pancreatic tissue, so that the pancreatic tissue fibrosis is degraded, the pancreas function is restored indirectly.

The green tea EGCG pretreated stem cells (DM+E-ADSC) protect the pancreatic tissue better than the untreated stem cells (DM+ADSC). It is found in the cell experiment that the survival related protein p-Akt of stem cells is scaled down in high glucose environment, so that the viability of stem cells in high glucose environment decreases. The p-Akt of stem cell is scaled up after the green tea EGCG pretreatment, so that the cell viability of stem cells increases in high glucose environment. According to the experimental results, when the stem cells are transplanted to the diabetic animal, the high glucose environment resulted from diabetes scales down the stem cell survival p-Akt, so that the viability of stem cells in the diabetic animal body is reduced, and the treatment effect is degraded. If the stem cells are pretreated with EGCG before the transplantation to the diabetic animal for treatment, the p-Akt survival is scaled up, so that the resistance of stem cells to the high glucose environment caused by diabetes is enhanced (EGCG pretreated stem cells are unlikely to die in high glucose environment), and then there are considerable stem cells survive the diabetic animal body, certainly the function to protect (repair) pancreatic tissue is better.

The following key points are derived from experimental conclusions: (1) the EGCG pretreatment can scale up the resistance of stem cells to high glucose environment; (2) the EGCG treated stem cells have better abilities to repair the damaged pancreatic tissue and to reduce blood glucose than the stem cells without any treatment; (3) the EGCG treated stem cells repair the damaged pancreatic tissue by scaling up the survival related protein in pancreatic tissue, scaling up the Sirt1 pathway related protein, scaling down the apoptosis related protein and scaling down the fibrosis related protein.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method for repairing a damaged pancreas in a subject, wherein the method comprising administrating to said subject a plurality of pretreated adipose-derived stem cells and an ester type catechin, wherein the plurality of pretreated adipose-derived stem cells are cultured with the ester type catechin.

2. The method of claim 1, wherein the damaged pancreas cause diabetes or hyperglycemia.

3. The method of claim 1, wherein the damaged pancreas is a plurality of damaged beta cells.

4. The method of claim 1, wherein the pretreated adipose-derived stem cells are pretreated with the ester type catechin for 2 hours.

5. The method of claim 1, wherein the concentration of ester type catechin for the pretreated adipose-derived stem cells is 5-25 μM.

6. The method of claim 1, wherein the concentration of ester type catechin for the pretreated adipose-derived stem cells is lower than 20 μM.

7. The method of claim 1, wherein the pretreated adipose-derived stem cells have higher viability in a high glucose environment, wherein the high glucose environment means the glucose concentration is 22 mM˜44 mM, mainly 33 mM, the high viability is 80%˜100%.

8. The method of claim 1, wherein the pretreated adipose-derived stem cells increase expression level of a group of survival related protein in the damaged pancreas, wherein the group of survival related protein comprising p-IGF1R, PI3K, p-PI3K, Akt, p-Akt, p-Bad and Bcl-xL, the percentage of increase is 50%˜100%.

9. The method of claim 1, wherein the pretreated adipose-derived stem cells increase expression level of a group of Sirt1 pathway related protein in the damaged pancreas, wherein the group of Sirt1 pathway related protein comprising p-AMPK and Sirt1, the percentage of increase is 50%˜100%.

10. The method of claim 1, wherein the pretreated adipose-derived stem cells reduce expression level of a group of cell apoptosis pathway related protein in the damaged pancreas, wherein the group of cell apoptosis pathway related proteins comprising FADD, Caspase 8, Bax, Cytochrome C and Caspase 3, the percentage of decrease is 40%˜80%.

11. The method of claim 1, wherein the pretreated adipose-derived stem cells reduce expression level of a fibrosis related protein in the damaged pancreas, wherein the fibrosis related protein is TGF-β, the percentage of decrease is 40%˜80%.
